(19) United States
(12) Patent Application Publication
(10) Pub. No.: US 2025/0325209 A1
(43) Pub. Date: Oct. 23, 2025

(54) SYSTEM AND METHOD FOR MONITORING UROLOGY DISEASES AND SYMPTOMS

(71) Applicant: CARETECH HUMAN INC., Lewes, DE (US)

(72) Inventor: Denys Vladymyrovych Matsui, Lewes, DE (US)

(21) Appl. No.: 18/866,933

(22) PCT Filed: May 18, 2023
(86) PCT No.: PCT/US23/22701

§ 371 Date: Nov. 18, 2024

Related U.S. Application Data
(60) Provisional application No. 63/343,113, filed on May 18, 2022.

(51) Int. Cl. A61B 5/20; A61B 5/00; A61B 5/117; G16H 10/60

(52) U.S. Cl. CPC A61B 5/208; A61B 5/117; A61B 5/6891; A61B 5/7275; G16H 10/60

(57) ABSTRACT

A system for monitoring urological diseases and symptoms including a sampling reservoir having an opening and having a liquid held therein, the sampling reservoir configured to receive a biomaterial sample from a user through the opening, a sensor disposed proximate to the sampling reservoir, the sensor configured to measure a parameter of the liquid, generate a signal in response to the measurement, and a computing node, the computing node configured to receive the signal from the sensor, and determine therefrom a parameter of the biomaterial sample.

### CROSS REFERENCE TO RELATED APPLICATIONS

This application claims the benefit of U.S. Provisional Application No. 63/343,113, titled “A SYSTEM AND METHOD FOR UROLOGY DISEASES AND SYMPTOMS MONITORING,” filed on May 18, 2022, the entire contents of which are hereby incorporated by reference herein.

### BACKGROUND OF THE DISCLOSED SUBJECT MATTER

### Field of the Disclosed Subject Matter

The disclosed subject matter relates to a system for disease and symptom monitoring. Particularly, the present disclosed subject matter is directed to a system and method for urology diseases and symptoms monitoring.

### Description of Related Art

Urologic diseases include a wide variety of conditions, all related to the filtering and carrying of urine out of the body. These diseases can affect men, women, and children of all ages.

There are many urologic disorders and diseases, including Benign Prostatic Hyperplasia (BPH), Urinary Incontinence, Urinary Tract Infections (UTIs), Overactive Bladder (OAB), Kidney and Ureteral Stones, Prostate Cancer, Bladder Cancer, etc.

The global prevalence of most mentioned diseases is growing dramatically. For instance, as many as 14 million men in the United States have symptoms of BPH. Roughly 40 percent of females and 12 percent of males have a UTI that causes noticeable symptoms. 15 million people in the United States have incontinence.

In particular, urology telemedicine grew dramatically in 2020-2021. But the instruments to support this transition are almost absent. In the urology domain, there are no analogies to blood pressure devices, thermometers, or sugar level analyzers. Telehealth consultations and follow-on recommendations are based on subjective patient complaints, leading to ineffective management of health conditions.

The same problem even with in-office visits. To perform first-level diagnostics (health screening) the urologists use various surveys including International Prostate Symptoms Score (IPSS) and uroflowmetry test (urodynamic). IPSS is an eight-question written screening tool used to screen for, rapidly diagnose, track the symptoms, and suggest management. It is widely used by urologists however it is time-consuming and super subjective. Uroflowmetry is a test that measures the volume of urine released from the body, the speed with which it is released, and how long the release takes. The in-office uroflow test is stressful. Many patients find it difficult to perform it in an artificial “high pressure” environment. In addition to that, it is proven that periodic one-time in-office uroflowmetry does not provide reliable patient data and, therefore, it may mask a patient's actual symptoms. A single in-clinic test can obtain a result within 50% accuracy and often fails to show changes in a patient's symptoms over time.

Efficient monitoring of Lower Urinary Tract Symptoms (LUTS) can help to alleviate urology disease prevalence, facilitate early detection, improve treatment outcomes and increase the quality of life globally. It can support the important trend in healthcare, namely the shift from sickness care to wellness care. In other words, changes in health management from yearly health checks to continuous monitoring. The modern approach is to catch diseases even before symptoms occur, and obviously, single data points during in-office visits are not sufficient anymore. This trend facilitated the rapid telemedicine boom and virtual-first concept that we've evidenced recently.

So currently, there is a significant technology gap in urology screening both in-home and in-office. Urologists are left behind from modern trends and lack instruments to follow best practices implementation. This is one of the largest unmet need in all of healthcare.

To tackle this challenge there are several types of products and concepts including app-based diaries, toilet superstructures, manual gadgets, and in-office uroflowmetry machines.

For instance, U.S. Pat. No. 2020/0054265A1, and WO2008030692A2 present a wide list of patents describing a computer-based system detecting and calculating urination sound. This can be used for urination intensity estimations but has several disadvantages related to expensive sound processing and the significant influence of distance and personal urination habits.

U.S. Pat. No. 20,211,77329A, DE102014008760B4, CA3102218A1, CN2221776Y represent a description of “pee-through” devices that can measure the intensity of urination, volume, and maximum speed. These solutions are based on weight measuring technologies or flow dynamics principles. Some of them a user has to hold in their hand while urinating. Most of the solutions require regular cleaning. The biggest disadvantage is that a user should install/turn on/activate, prior to each urination, which is a big deal if a person has urgency symptoms. Human factors can lead to mistakes and data inconsistency, amongst other issues. The complexity of the technology, size, weight, and cost are also disadvantages.

There is a technology gap in urology symptoms and disease screening both in-home and in-office settings. Current solutions require complex interaction of a user with a device, including the need for a person to perform an action to launch, control, hold, initiate, clean or calibrate diagnostic/screening tools to perform. Thus existing solutions are inconvenient, unreliable, difficult to clean, and stressful and health data are unreliable. As a result, doctors are operating with inconsistent and subjective data, which obviously badly influences the quality of medical decisions.

There thus remains a need for an efficient and economic method and system for urology diseases and symptoms monitoring.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a system for monitoring urological diseases and symptoms including a sampling reservoir having an opening and having a liquid held therein, the sampling reservoir configured to receive a biomaterial sample from a user through the opening, a sensor disposed proximate to the sampling reservoir, the sensor configured to measure a parameter of the liquid, generate a signal in response to the measurement, and a computing node, the computing node configured to receive the signal from the sensor, and determine therefrom a parameter of the biomaterial sample.

The disclosed subject matter also includes a method for monitoring urological diseases and symptoms including receiving a biomaterial sample stream from a user at a sampling reservoir having a liquid therein, measuring a parameter of the liquid within the sampling reservoir via at least one sensor, generating a signal in response to the measurement of the parameter of the liquid within the sampling reservoir, receiving, via a computing node, the signal from the sensor and determining therefrom a parameter of the biomaterial sample stream, and generating, via the computing node, an electronic diary entry in response to the signal.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic representation of the system for urological disease and symptoms monitoring in accordance with the disclosed subject matter.

FIG. 2 is a schematic view of an exemplary embodiment of the system for urological disease and symptoms monitoring shown in FIG. 1.

FIG. 3 is a schematic cross sectional view of a sampling reservoir in accordance with the disclosed subject matter.

FIGS. 4A-4B are schematic representations of the functionality of sensors measuring amplitudes of waves in the liquid in the sampling reservoir in accordance with the disclosed subject matter.

FIGS. 5A-5C are representations of an embodiment of the system for monitoring urological diseases and symptoms in accordance with the disclosed subject matter.

FIG. 6 is a schematic representation of a sensor suite in accordance with the disclosed subject matter.

FIG. 7A-7D are representations of plots of biological sample deposition in a sampling reservoir in accordance with the disclosed subject matter.

FIG. 8 is a schematic representation of a calibration system in accordance with the disclosed subject matter.

FIG. 9 is a flowchart representing a method for monitoring urological diseases and symptoms in accordance with the disclosed subject matter.

FIG. 10 depicts a computing node according to an embodiment of the present disclosure.

### DETAILED DESCRIPTION OF AN EXEMPLARY EMBODIMENT

Reference will now be made in detail to exemplary embodiments of the disclosed subject matter, an example of which is illustrated in the accompanying drawings. The method and corresponding steps of the disclosed subject matter will be described in conjunction with the detailed description of the system.

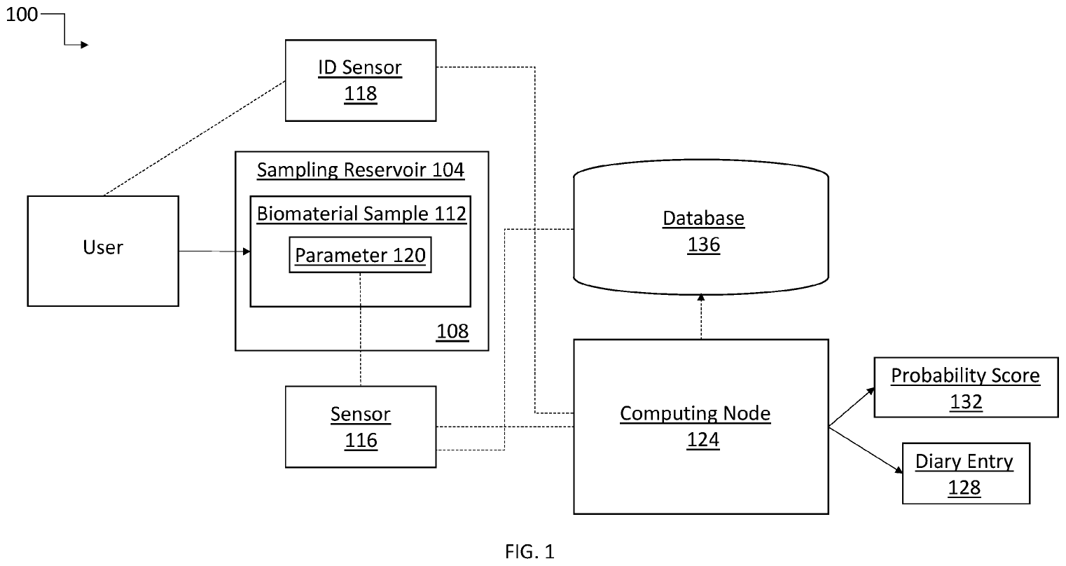

The methods and systems presented herein may be used for disease and symptom monitoring. The disclosed subject matter is particularly suited for urology and other diseases and symptoms monitoring. For purpose of explanation and illustration, and not limitation, an exemplary embodiment of the system in accordance with the disclosed subject matter is shown in FIG. 1 and is designated generally by reference character 100. Similar reference numerals (differentiated by the leading numeral) may be provided among the various views and Figures presented herein to denote functionally corresponding, but not necessarily identical structures.

As shown in FIG. 1, the system 100 for monitoring of urological diseases and symptoms is shown in schematic view. System 100 is configured to provide an uroflowmetry (urodynamic) solution, having at least one of a device and software, and configured to measure how fast urine flows, how much flows out, and how long it takes. System 100 may be operated in a majority automated regime, operating at least partially autonomously. System 100 may be integrated into one or more users' everyday routine activities without additional actions, utilization habits and/or activation—thereby making the system 100 extremely streamlined and repeatedly usable. In various embodiments, system 100 may be formed from custom or off-the-shelf components, as will be described in detail herein below, thereby making system 100 affordable or cost-efficient in a plurality of situations, such as home use, office use, public use, or medical use cases. In various embodiments, system 100 may be configured to enable continuous health measurements and vivid urology health analytics both for user and doctor, contemporaneously or stored in a database, as will be described herein. In various embodiments, system 100 may be configured to detect, measure and track urological and other types of diseases, disorders, illnesses, or symptoms that may lead or diagnose said diseases, disorders, and illnesses, such as LUTS, including symptoms in voiding urine and the urinary tract. In various embodiments, system 100 may be a single use system, configured to be discarded after use. In various embodiments, system 100 or any component thereof, may be configured to be cleaned between uses, sets of uses, periods of time, between users, or another factor. In various embodiments, system 100 or any component thereof may be configured to self-clean or be cleaned by the flushing of the toilet (water rushing from the toilet tank or rerouted therefrom). In various embodiments, system 100 or any component thereof may be formed integral to a unit of furniture or room, such as installed within a wall or ceiling or toilet/toilet tank. In various embodiments, system 100 may measure one or more parameters associated with uroflowmetry. In various embodiments, system 100 may measure one or more parameters associated with IPSS, voiding characteristics (such as volume and frequency), foaming and/or chemical makeup of the biomaterial sample. System 100 may be utilized within one voiding episode or over any number of voiding episodes.

Referring to FIG. 1, system 100 for monitoring urological diseases and symptoms includes a sampling reservoir 104. Sampling reservoir 104 having at least one opening therein. Sampling reservoir 104 configured to hold a liquid 108 therein, the liquid 108 held in a still or stable environment to avoid ripples or waves in the liquid 108. In various embodiments, the liquid 108 may be exposed to the exterior of sampling reservoir 104 proximate the opening in the sampling reservoir 104.

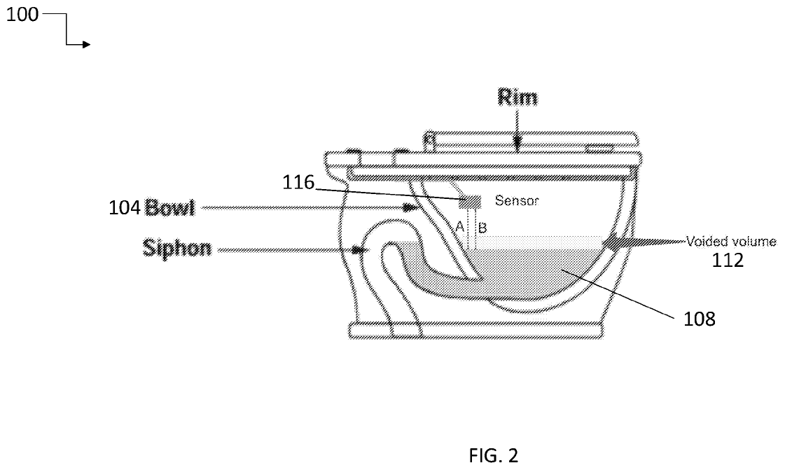

In various embodiments, sampling reservoir 104 may be a toilet bowl. In various embodiments sampling reservoir 104 may be a urinal. In various embodiments, sampling reservoir 104 may be a bidet. In various embodiments, sampling reservoir 104 may be a bedpan or medical container. In various embodiments, sampling reservoir may be a tank, jug or bottle fitted as will be described herein. In various embodiments, sampling reservoir 104 may be disposed in a municipal building bathroom (restroom). In various embodiments, sampling reservoir may be a residential home's toilet, such as a siphonic toilet like in FIG. 2. In various embodiments, sampling reservoir 104 may be a commercial building's toilet. Sampling reservoir 104 may be hemispherical in shape, having a hollow cavity, wherein the circular or generally oblong flat surface is a rim. In various embodiments, sampling reservoir 104 may include a seat disposed proximate the opening, such as a seat with an opening abutting the rim. In various embodiments, the seat may be a toilet seat in a residential toilet. In various embodiments, system 100 may be disposed on a plurality of toilets, each toilet configured and equipped to measure urine entering the bowl. For example and without limitation, there may be a plurality of toilets that a single user uses. Each toilet can be equipped with or form a portion of system 100. In various embodiments, any data measured, input by the user, or scores or diary entries formed therefrom may be aggregated in the cloud and linked to the single person. In this configuration, a single user can use a plurality of toilets, the system attached to said toilet being able to identify said user, and aggregate the user's data across the plurality of toilets.

In various embodiments, liquid 108 may be water. In various embodiments the water may be supplied by an onsite tank or provided by one or more offsite locations such as municipal utilities or another exterior source. Liquid 108 may be natural water or include one or more additives dispersed in solution therein. In various embodiments, liquid 108 may be any liquid having a density and viscosity, wherein the liquid 108 may have predictable waves, perturbations, disturbances, or ripples imparted thereto by physical interference with another liquid stream coming into contact with the standing liquid 108.

With continued reference to FIG. 1, sampling reservoir 108 may be configured to receive a biomaterial sample 112 from a user. Biomaterial sample 112 may be formed as a liquid. In various embodiments, biomaterial sample 112 may be a solid or a gas. In various embodiments, biomaterial sample 112 may be a mixture of material in various phases of matter. In various embodiments, biomaterial sample 112 may be urine voided from a user into the sampling reservoir 104 through the opening and into the liquid 108. In various embodiments, biomaterial sample 112 may be solid excrement, gaseous excrement or other material voided by a biological body, such as a human user. Biomaterial sample 112 may be deposited by a user into sampling reservoir 104 while sitting, standing, kneeling, or another position. Biomaterial sample 112 may be a stream of urine having a flow rate. Biomaterial sample 112 may be a stream of urine having a mass flow rate. Biomaterial sample 112 may be a stream of urine having a flow pattern. Biomaterial sample 112 may be deposited over a period of time, hereinafter referred to as the void time. In various embodiments, biomaterial sample 112 may be deposited in one or more periods of time, the total or episodic elapsed times for deposit may be tracked by the system as described herein. In various embodiments, biomaterial sample 112 has a void volume, wherein the volume of voided material is added to the liquid 108 within the sampling reservoir. Biomaterial sample 112 may have a void frequency, tracking the amount of times, time in between or other frequency related parameter of the user depositing the biomaterial sample 112 into one or more sampling reservoirs 104.

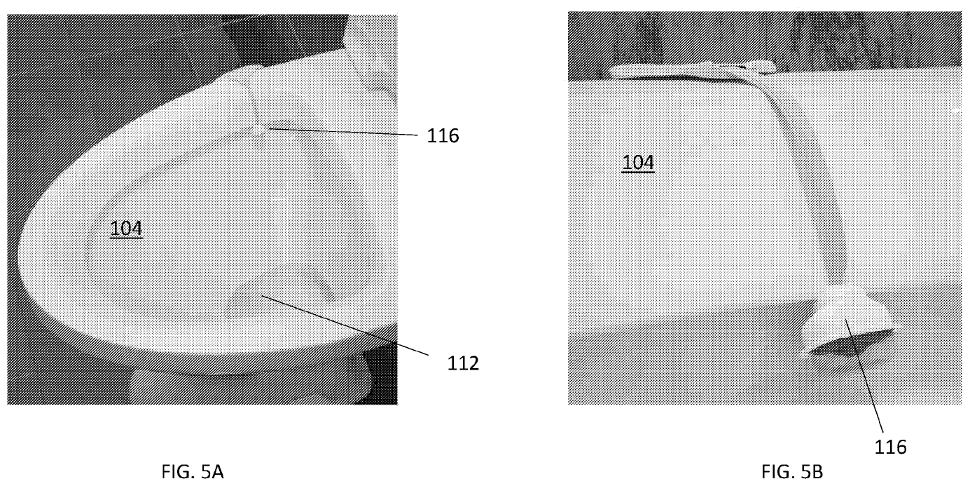

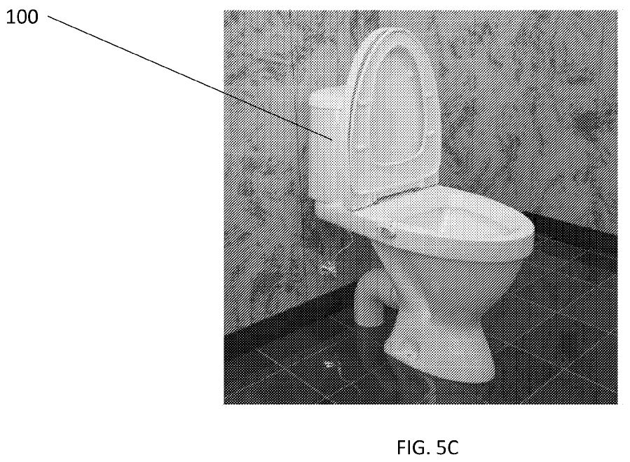

With continued reference to FIG. 1, system 100 includes a sensor 116. Sensor 116 may be an optical sensor or an optical sensor-based technology, as will be described herein. Sensor 116 may be any sensor configured to detect or measure a parameter it is in physical contact with. Sensor 116 may be any sensor configured to detect or measure a parameter it is not in physical contact with, thus detecting said parameter from a distance. Sensor 116 may be coupled to sampling reservoir 104. In various embodiments sensor 116 may be coupled to the rim portion of a toilet bowl, as can be seen in FIGS. 5A-5C. Sensor 116 may emplaced on the toilet tank, behind said toilet bowl. Sensor 116 may be configured to aim at the toilet bowl from the wall or toilet tank, when not emplaced within or proximate the opening of sampling reservoir 104. In various embodiments, sensor 116 may include one or more gyroscopic or GPS sensors configured to provide the system 100 or the user with location information of the sensor 116, including location on the toilet bowl and orientation of the sensor itself relative to the liquid 108. Sensor 116 may be coupled to the sampling reservoir 104 proximate the opening, the sensor 116 configured to detect or measure a parameter of the liquid 108 disposed within the sampling reservoir 104. In various embodiments, sensor 116 may be coupled to the sampling reservoir 104 proximate the opening, the sensor 116 configured to detect or measure a parameter of the biomaterial sample 112 entering the sampling reservoir 104 through the opening. In various embodiments, sensor 116 may be powered by a battery, such as AAA batteries, AA batteries or rechargeable batteries, such lithium ion and/or iron-chloride batteries.

Sensor 116 may be coupled to the sampling reservoir 104 proximate the opening of the sampling reservoir and extending inward to be disposed over the liquid 108, as can be seen in FIG. 2. In various embodiments, sensor 116 may be disposed laterally in the sampling reservoir relative to the user, being disposed on the left or right side of the sampling reservoir, such as can be seen in FIGS. 5A-5C. Sensor 116 may be disposed longitudinally relative to the user, such as at a portion of the sampling reservoir 104 proximate the user or across the bowl from the user, the latter case can be seen in FIG. 2. And further in FIG. 2, sensor 116 may measure distances A and B, the distances between the sensor 116 to the liquid prior and after deposition of a voided volume of a biomaterial sample 112. In various embodiments, sensor 116 may be emplaced above the liquid 108 and configured to view said liquid 108 at a normal angle. In various embodiments, sensor 116 may be configured to produce an optical signal, such as a beam, direct it at the liquid 108, and measure one or more optical phenomena reflected back the sensor. In various embodiments, sensor 116 may be disposed at some point along the upper portion of the bowl proximate the rim portion at some point within the concave bowl of sampling reservoir 104. In various embodiments, sensor 116 may be emplaced underneath the liquid 108 line of the sampling reservoir 104. In various embodiments, sensor 116 may be submerged under the water of the toilet bowl, the sensor aimed upward at the surface of the water therein. In various embodiments, sensor 116 may be emplaced at the waterline of liquid 108 within the sampling reservoir 104.

Sensor 116 may be configured to measure or detect one or more parameters 120 associated with LUTS. LUTS are the symptoms of many urology diseases. LUTS can include irritative (urgency, frequency, nocturia), obstructive symptoms (hesitancy, a weak and interrupted urinary stream, straining to initiate urination, a sensation of incomplete bladder emptying) and urinary incontinence. Parameter 120 may be one or more physical characteristics of the stream of biomaterial sample 112. Parameter 120 may be flow rate, mass flow rate, volumetric flow rate or a similar characteristic of the flow of biomaterial sample 112 from the user. In various embodiments, sensor 116 may be configured to measure parameter 120 related to an average characteristic, such as average flow rate (Q_{avg}), maximum flow rate (Qₘₐₓ) and abnormal flow patterns. In various embodiments, flow rate, flow consistency or other parameter 120 associated with urination may be captured within parameter 120. Sensor 116 may be configured to measure a flow rate of urine into the sampling reservoir 104. Sensor 116 may be configured to detect the entrance of biomaterial sample 112 into sampling reservoir 104. In various embodiments, sensor 116 may be configured to measure a volume of deposition of biomaterial sample 112 into sampling reservoir 104. For example and without limitation, sensor 116 may be configured to measure a volume or level of liquid 108 in the sampling reservoir 104 prior to deposition of biomaterial sample 112, and measure the volume or level of liquid 108 after deposition of biomaterial sample 112. Sensor 116 may be fed the volume or measurements of the sampling reservoir 104, thereby providing the capability to one or more processors communicatively connected thereto to measure the volume of biomaterial sample 112 deposited therein.

Additionally or alternatively, the volume of excreted urine may be calculated by converting the intensity of the waves measured by the sensor 116 into one or more quantitative values. Two or more methodologies may be utilized separately, sequentially, contemporaneously or partially supporting each other.

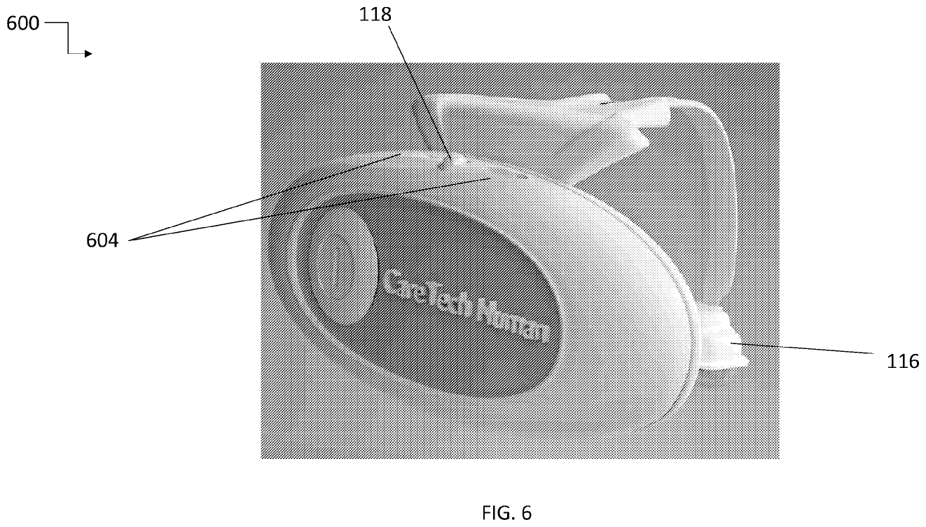

The user may input their height, providing system 100 (and more specifically computing node 124) the height at which the user urinates. The system 100 may guide the user via a plurality of possible means e.g., by blinking LEDs, smartphone applications, or sound commands. For example and without limitation, system 100 may visually indicate to user via visual device 604, which may be blinking, colored, or otherwise illuminated LEDs, as shown in FIG. 6. For example and without limitation, one or more LEDs may emit a red color when system 100 is in one or more modes, such as a calibration mode. For example and without limitation, one or more LEDs may emit a green color when connected to a wireless internet connection, such as a cellular network or WiFi. For example and without limitation, one or more LEDs may emit a third color, cease emitting light, or produce audio signals such as beeps, when the system 100 transmits data to one or more computing devices 124 or the cloud. The user is directed to use a sampling reservoir 104 such as the toilet bowl, beaker, medicine glass (or any other product providing similar functions) and pour water into the toilet bowl from the user's normal urination height. During this process, the sensor 116 obtains wave intensity readings from liquid 108's surface within the toilet bowl and associates them with the poured volume. By repeating the described procedure one or more times, the system 100 and specifically, sensor 116 gains calibration data. Every further urination, this data will be used to calculate voided volume (as a parameter 120) based on measured waves intensity in the toilet bowl.

Additionally or alternatively, the user may input his/her height, so the system 100 may infer at what height the user urinates. The system 100 may then guide the user via any possible means e.g., by blinking LEDs, smartphone applications, or sound commands, such as visual signal 604 to initiate deposit of the biomaterial sample 112. The user is directed to use the sampling reservoir 104, toilet bowl, beaker, medicine glass (or any other product providing similar functions) and pour water into the toilet bowl. The sensor 116 measures the distance to the liquid 108's level before and after pouring the water into the toilet bowl. The calculated difference between the initial and eventual liquid 108 level within the sampling reservoir 104, for example, the toilet bowl model/manufacturer will be strictly proportional to the amount of poured water. By repeating the procedure any number of iterations, system 100 and sensor 116 gains calibration data. This data can be used every urination to calculate voided volume based on measured waves intensity in the toilet bowl.

The user may be prompted to input the specific sampling reservoir 104, such as the toilet bowl manufacturer and model. Database 136 may store calibration information related to any number or variety of sampling reservoirs 104, such that the user only needs to input the toilet bowl model into the solution interface and optionally can bypass calibration step before using the system 100.

In various embodiments, the system 100 may require calibration, for example and without limitation, during the urination process urine stream falls onto the water inside the toilet bowl. It generates waves on the surface of the water in the toilet bowl. The height and/or amplitude of the waves are proportional to the intensity or flow (e.g. ml/sec) of the urination. The detection of waves' height/amplitude can be performed by a light-emitting sensor as described herein. The optical sensor, which in embodiments, is a TOF sensor or camera, may measure distance by actively illuminating an object with a modulated light source such as a laser and a sensor that is sensitive to the laser's wavelength for capturing reflected light. The sensor measures the time delay between when the light is emitted and when the reflected light is received by the camera and thus can calculate the distance to the object or the distance to the wave crest or to the bottom of the wave. Using this principle, it is possible to calculate the height/amplitude of the waves, which is proportional to the intensity of urination—thereby measuring a parameter 120 of the biomaterial sample 112.

In various embodiments, sensor 116 may be an optical sensor, configured to detect or measure an aspect of electromagnetic radiation. In various embodiments, sensor 116 may be configured to measure or detect a characteristic of visible light. In various embodiments, sensor 116 may be configured to measure or detect a characteristic of invisible light, such as infrared, near infrared and/or ultraviolent light. In various embodiments, sensor 116 may be a time-of-flight (TOF) sensor. In various embodiments, the TOF sensor may be a ST VL53L4CD type sensor or a similar sensor.

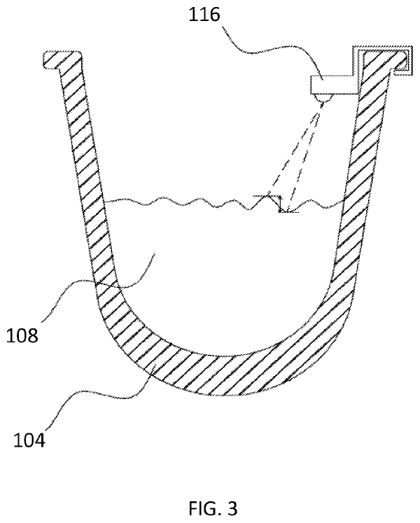

Referring to FIG. 3, system 100 includes sensor 116 that is configured to detect the timeframe to sample biomaterial sample 112. During the urination process urine stream falls into the water inside the toilet bowl. It generates waves on the surface of the water in the toilet bowl. The decrease in urination leads to a decrease in the intensity of waves inside the toilet bowl and vice-versa. The decrease in waves or their absence can indicate the moment when urination is completed. The detection of waves can be performed with mentioned sensors such as sensor 116 disposed on the side of the toilet bowl as shown, or another sensor or plurality thereof. Using this principle, and with mentioned types of sensors, it is possible to detect the optimal timeframe for sampling. According to embodiments of the disclosed subject matter, the one or more sensors configured to determine the timeframe to sample for biomaterial sample 112 may be separate and standalone to any of the described sensors configured to extract data and may be communicatively coupled thereto. For example and without limitation, sensor 116 as shown in FIG. 3 may include a distinct sensor such as a time-of-flight sensor configured to determine the amplitude of waves in the bowl and communicate to another distinct sensor to measure some parameter 120 of the biomaterial sample 112 there deposited.

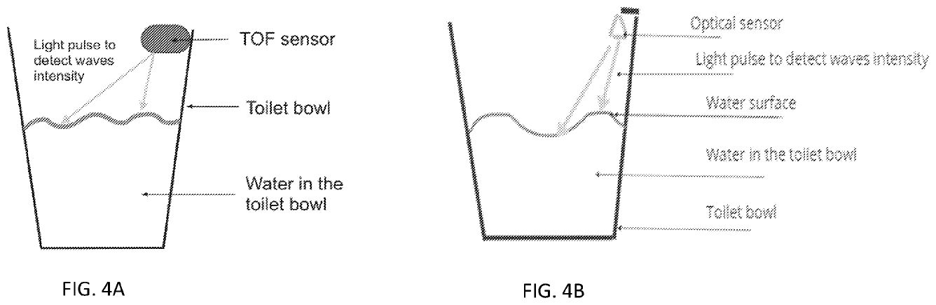

In various embodiments, sensor 116 may be a triangularity sensor. In various embodiments, the triangularity sensor is configured to measure a distance from the sensor itself to the water line or some portion of the liquid 108 in the sampling reservoir 104. In various embodiments, triangularity sensor may be configured to measure an intensity of a wave in the liquid 108 created by the biomaterial sample 112 as shown in FIGS. 3 and 4A-4B. In various embodiments, triangularity sensor may be a Sharp GP2Y0A41SK0F. In various embodiments, sensor 116 may be a proximity sensor consistent with any description of a proximity sensor herein, such as a Vishay VCNL4040 with one or more light emitters operating in various wavelengths of light. In various embodiments, any sensor 116 as described herein may be configured to operate in the frequency regime from 2-400 Hz. In various embodiments, any sensor 116 (or ID sensor 118, below) may operate in any frequency range known in the art.

For example, and without limitation, system 100 can include one or more optical sensors, such as one or more of a Sharp GP2Y0A41SK0F or Vishay VCNL4040. Those sensors may utilize one or more artificial light signals and measures one or more distances between the camera and the subject. In this non-limiting embodiment, the subject is water in the toilet bowl and its movements or fluctuations. The specific light length may be around 900 nm with an emitter angle of about 7-60 degrees may allow for receiving reflected light and detecting signal readings from the liquid mixture inside the toilet bowl (water and urine). In various embodiments, the specific light length and emitter angle allow receiving reflected light in the ranges of 7-15 degrees, sensor may be oriented normally to the surface of the water.

During the urination process urine stream falls onto the water inside the toilet bowl. It generates waves on the surface of the water in the toilet bowl. The height and/or amplitudes of the waves captured by optical sensors strongly correlates with the urination (flow) pattern which can be seen in FIGS. 3 and 4A-4B.

In addition to the process described above, siphon-type toilet bowls (the type shown in FIG. 2) provide an opportunity to additionally measure (and/or adjust) the total voided biomaterial sample volume readings. The one or more sensors may measure the one or more distances to the water surface before and after the urination and recalculates the distance change to the voided volume, thereby measuring one or more parameters 120 as a result. This example may be seen in FIG. 2, wherein the voided volume of biomaterial sample 112 provides additional volume to the liquid 108 held in the sampling reservoir.

With continued reference to FIG. 1, system 100 includes an identification (ID) sensor 118. ID sensor 118 may be a proximity sensor as described herein. ID sensor 118 may be disposed proximate the sampling reservoir 104 and configured to have a field of view surrounding the sampling reservoir 104. In various embodiments, ID sensor 118 may be emplaced exterior to the sampling reservoir 104, for example and without limitation, emplaced on a wall proximate the toilet. In various embodiments, ID sensor 118 may be emplaced on the sensor 116, on the sampling reservoir 104, itself, or another portion of system 100. In various embodiments, ID sensor 118 may be configured to detect the user approaching the sampling reservoir 104. In various embodiments, ID sensor 118 may be configured to generate a signal in response to detection of a user. ID sensor 118 may transmit said signal to one or more other components of system 100, namely sensor 116, computing node 124 or database 136 in response to the detection. In various embodiments, ID sensor 118 may generate a signal in response to the detection, the signal configured to initiate a function of the component, for example and without limitation, activate sensor 116, activate computing node 124, or store the detection as data within database 136. In various embodiments, ID sensor 118 may be configured to receive identification information via one or more user inputs, such as buttons, fingerprints, voice commands, or measured parameters 120 (via sensor 116 or directly measured via ID sensor 118). In various embodiments, ID sensor 118 may be configured to receive identification information from a user device such as a smartphone, smart watch, or wearable technology device via Bluetooth or Non-Fungible Token (NFT).

In various embodiments, ID sensor 118 may be configured to identify the user as an individual. For example and without limitation, identifying the user may include comparing characteristics of the user with stored data in the database 136. ID sensor 118 may be a biometric scanner, configured to measure a user's body or face to identify the user. In various embodiments, ID sensor 118 may measure a parameter 120 of the biomaterial sample 112 to identify the user. In various embodiments, ID sensor 118 includes a fingerprint scanner. In various embodiments, system 100 may be configured to re-identify a user in a manner disclosed in International Application PCT/US2022/033838, titled

“IDENTIFICATION OF A PERSON BY THE UNIQUE CHEMICAL COMPOSITION OF A BIOMATERIAL IN DIFFERENT PHASES” the entirety of which is hereby incorporated by reference herein.

In various embodiments, ID sensor 118 may be configured to measure, detect or receive user inputs regarding optional or manual information related to urological diseases/conditions and/or symptoms. For example and without limitation, ID sensor 118 may be a keyboard, microphone or other input apparatus for a user to input subjective or qualitative information regarding feelings related to the urological symptoms. The qualitative user data may be transmitted to one or more of the computing node 124, cloud computing environment, database 136 and/or sensors for analysis.

Additionally or alternatively, ID sensor 118, sensor 116 or another sensor or sensor suite may be configured to detect or measure a parameter of the user. For example and without limitation ID sensor 118 (or the other sensors) may measure the speed at which the user approaches the sampling reservoir 104. In various embodiments, ID sensor 118 may measure or detect the movement of the user. ID sensor 118 may transmit said data to the computing node 124, said computing node 124 configured to predict or estimate the urgency at which the user is approaching the sampling reservoir 104, the delay or urgency with which the user starts depositing the biomaterial sample (urinating), detect whether the user is sitting or standing. In various embodiments, sensor 116 may be configured to detect if the user is sitting on the toilet, in which case the toilet bowl will be darker. In various embodiments, sensor 116 may be configured to detect if a person is standing, wherein the toilet bowl is brighter, having generally the light of the room entering the toilet bowl, and in embodiments, shadowed by the standing user. In various embodiments, the ID sensor 118 may transmit said data to the computing node 124 in order for the computing node 124 to determine whether the user is having a defecation episode, thereby labeling any other data collected as such, as unusable, or to deactivate any electrical components. Any sensor as described herein may be configured to communicate, or commonly transmit data to the computing node 124 to perform the same or similar functions. In various embodiments sensor 116 may include a wired connection to any other component in system 100, such as computing node 124 and configured to transmit electrical signals through said wired connections. In various embodiments, any component of system 100 may be connected via a USB cable. In various embodiments, sensor1 116 may include an antenna or other component configured to transmit electrical signals or data over a wireless connection to one or more of computing node 124, database 136 or ID sensor 118. For example and without limitation, sensor 116 may transmit data or signals to computing node 124 via a telecommunications link such as 2G, 3G, 4G, 5G, over a WiFi connection, and/or a Bluetooth connection. In various embodiments, one or more sensors 116 may communicate with computing node 124 over an Internet connection.

In various embodiments, sensor 116 and computing device 124 may detect bubbles on the water surface that alter the signal by modifying the spectral set of harmonics. System 100 may operate under the same principles as described herein. In various embodiments, the same or separate algorithms (which work in tandem with the other algorithms) that detect “bubbling zones” in the signal readings and equalize them. Bubbling of urine is valuable diagnostic information per se as it may show protein present in urine due to kidney dysfunctions, high blood pressure, and other health conditions. In various embodiments, one or more data elements or extracted features may be representative of bubbles, which may be used as a quantitative feature for diagnosis. Additionally or alternatively, one or more predictive models may be capable of performing predictions and detecting bubbles simultaneously. In various embodiments, sensor 116 may identify the presence of bubbles in the urine-liquid 108 mixture. One or more components of the system 100 may intake the presence of bubbles from sensor 116 and adjust one or more parameters, feature extraction steps, predictive models or another component to account for the presence of bubbles and/or foam.

With continued reference to FIG. 1, system 100 includes a computing node 124. Computing node 124 may perform data processing functions, and may be formed by one or more computer devices, minimally equipped with at least one processor device, random access memory (RAM), and read-only memory as well as data input-output devices communicatively connected thereto or can be used as an external device. In embodiments, this device or plurality thereof, may be implemented and communicatively connected through the cloud. Also, it can be connected by data transmission channels to external data sources or external data processing resources. Computing node 124 may be disposed in, on, or nearby to system 100 and/or sampling reservoir 104 in the form of embedded hardware, software, or a combination thereof.

Data analysis can be performed using, for instance, but not limited to, machine learning (ML) predictive models based on modern neural networks as described herein below. In various embodiments, different types of neural networks, sequence-to-sequence encoders for signal, transformer neural network architecture and time series prediction may be employed in the processing of said data.

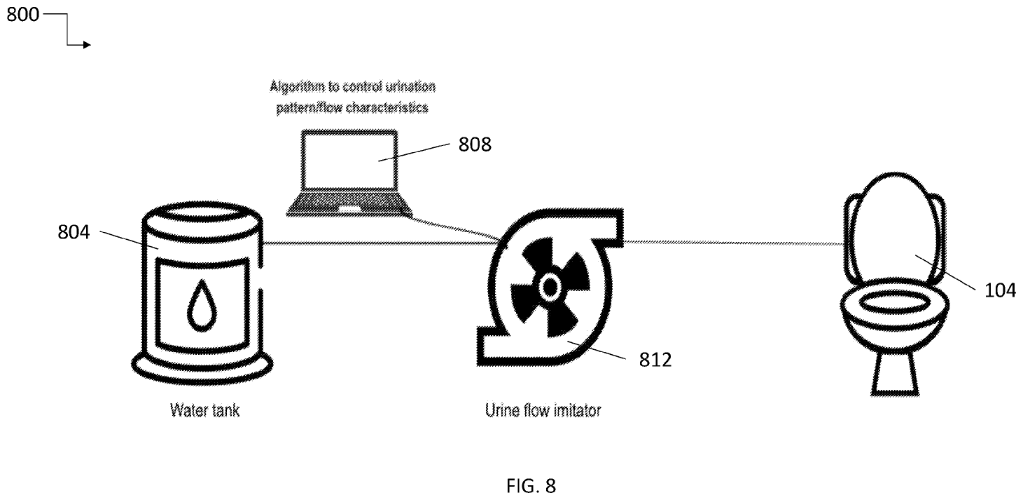

The initial data set can be obtained using real patient data coupled with referral information or using artificial voidings generated by a flow generator of any construction that can produce flow and measure its intensity using calibrated weights, a flowmeter, or any other calibration system, such as one shown in FIG. 8 described below.

Computing node 124 is configured to receive at least one datum of extracted data from the biomaterial sample in the form of one or more signals, electrical signals or the like. Computing node 124 may include, but is not limited to one or more devices having hardware and/or software configured for receiving, transmitting and storing data, which can be implemented using both wired and wireless data transmission technologies, including Wi-Fi technology, mobile radio communications using 2G, 3G, 4G, and 5G standards. The configuration of the data transfer device provides for the transfer of data either to one or more local processors, or to one or more cloud systems, or to one or more remote servers. Alternatively, the device for receiving, transmitting and storing data can be designed with the data processing device as a single device or can be integrated with any of the previously listed devices herein.

Computing node 124 includes hardware and/or software configured to extract a feature from the extracted data. The first feature may be the first of a plurality of extracted features, and in no way limits the number of collection of features intended to be extracted according to embodiments of the disclosed subject matter. The first feature may include a feature vector formulated from the extracted data according to the disclosed subject matter or another methodology. First feature may include one or more elements of computer-readable data, human readable data, matrices, listings of numbers, or the like. The first feature may include the results of one or more optimization problems, one or more coefficients of one or more polynomials and/or one or more roots of the one or more polynomials according to the disclosed subject matter. The first feature may include one or more unique parameters 120 and/or values that describe the flow of a biomaterial sample 112 or movement of liquid 108. The first feature may include numerical values representing macroscopic parameters corresponding to physical properties of biomaterial sample 112. The first feature may be computer interpretable or human interpretable. The first feature may include one or more parameters 120 representing a flow rate, volume, or other uroflowmetry parameter as described herein, in embodiments.

Such a feature extraction device can be integrated into the system 100 or analyzer or be used as an external device connected to the system 100 or to the analyzer by digital data transmission channels. Also, the feature extraction device can be connected by data transmission channels to external data sources or external data processing resources. Feature extraction can be processed on one or more devices having computing capacities, such as ESP 32 microcontroller, and/or transferred to remote computing capacities as described herein.

In various embodiments, features may include either processed or unprocessed measurements as discussed above. In addition, features may be extracted using feature extraction methods known in the art, such as PCA or autoencoders.

According to embodiments of the present disclosure, computing node 124 may include a plurality of subsystems, one of which may be a designated feature extraction system. For example and without limitation, the incoming extracted data for the feature extraction system can be a time series of measured values of, for example, distances measured by sensor 116 and identification information of ID sensor 118. Such influences may include, but are not limited, to sensor temperature, sensor voltage or sensor light exposure, among others. External influences are divided into one or more phases in which the dependence of the influencing quantity can be a constant or change according to a given law (e.g., influence modulation).

Computing node 124 then, from the extracted data, for each phase, or several phases together, features are extracted, according to the totality of which, for all phases or some of their subsets, a feature vector may built for further use in applications including but not limited to the user identification at a later date, disease detection and flow parameter classification, among other implementations.

According to embodiments, the feature may be presented in the form of a numerical vector that can be obtained from the input data, by means of transformation such as integral transformation, linear transformation, optimization problem solution, or transformation coefficients of which are determined as a result of the learning process, on a data sample.

In one approach computing node 124 may include a feature extractor that can be implemented as described in International Patent Application No. PCT/US2022/033838, the entirety of which is hereby incorporated by reference herein.

In another approach, computing node 124 may include a feature extractor that solves the optimization problem to fit the measured time series of one or more phases with a predefined function. The determined coefficients of the function or a subset thereof is used as a feature vector of different phases and may be used separately or combined into a joint feature vector.

In various embodiment, computing node 124 may include a feature extractor that uses machine learning to determine the transformation to be applied to input data to produce a feature vector. Either approach may be used alone or in combination. In various embodiments, the raw data received from the optical sensor can be transferred to the cloud for analysis. In one of the embodiments, the data extraction can have two phases. The first step of the data analysis may be a feature extraction and the second phase may be one or more machine learning processes.

During the feature extraction, the raw signal from one or more sensors 116 may be pre-processed to remove noise using low/high/band pass filters and optionally artifacts using a Kalman filter and then split into an array of signals by applying different band-pass filters to analyze different frequencies independently. A bandpass filter is applied to remove frequencies outside the range of interest. This can be achieved for example using the following formula for a butterworth bandpass filter:

$$H ( S ) = \frac{K}{{\prod }_{k = 1}^{n} {s}^{2} + {ω}_{k} s + {ω}_{k}^{2}}$$

where K is a constant, n is the filter order, s is the complex Laplace variable, and ωk is the cutoff frequency normalized by the sampling frequency fs.

Low cut and high cut in Hz are used to get corresponding ω:

$${ω}_{low} = \frac{{f}_{low}}{{f}_{N}} = \frac{2 {f}_{low}}{{f}_{s}}$$

$${ω}_{high} = \frac{{f}_{high}}{{f}_{N}} = \frac{2 {f}_{high}}{{f}_{s}}$$

Where Nyquist frequency ƒ_{N} is computed like this:

$${f}_{N = \frac{{f}_{s}}{2}}$$

Where ƒₛ is a sampling frequency. The output of the butterworth bandpass filter with coefficients b and a applied to the input signal x[n] is:

$$y [ n ] = {\sum }_{k = 0}^{M} {b}_{k} x [ n - k ] - {\sum }_{k = 0}^{M} {a}_{k} y [ n - k ]$$

where M is the length of the filter coefficients b, N is the length of the filter coefficients a, and y[n] is the output signal at time n.

From the filtered signal, various features are extracted for analysis. These can include but not limited to the frequency content of the signal in different frequency bands, the standard deviation of the signal, and normalized logarithmic sensor output to linear. The fast Fourier transform (FFT) is used to compute the frequency content of the signal. Using a sliding window of predefined size can be computed by applying filter with standard deviation kernel:

$${σ}_{i} = \sqrt[ ]{\frac{1}{N} {\sum }_{j = i - ω}^{i + ω} {( {y}_{i} - {μ}_{i} )}^{2}}$$

Where sigma is the standard deviation at position I, N, ƒ is the size of the window, yᵢ is the value of the input signal at position j, and μᵢ is the local mean position i, which is defined as

$${μ}_{i} = \frac{1}{N} {\sum }_{j = i - ω}^{i + ω} {y}_{i}$$

Where y is an input array, the output σ signal vector has the same length. This transformation can be executed in discrete or continuous space.

The features extracted in this step can be but are not limited to the initial distance to water before urination, water level after urination, the discretization parameter of sensor output, averaged signal for different frequency bands for a certain segment of signal, the dispersion (variance) between certain data points, smoothed dispersion, several signals with applied frequency filter, several signals with applied dispersion of frequency filter, exponential and logarithmic signals.

In some cases, there is a protein in the urine that leads to the creation of bubbles or foam on the water surface during the urination process. In this case, the water oscillation in the toilet bowl can be different. The experiment showed that the same approach as described above can be applied however equalizer may also be applied to the initial signal from the optical sensor.

In various embodiments, exploratory data analysis (EDA) can be used for extracting more informative signal features. The set of features can be used for further steps, such as algorithmic or ML-based information extraction. For example and without limitation, different frequency bands based deviations and signal averages, in some cases, can contain more information about the physical process of urination than full frequency range analysis. Also, an incremental average signal value growth may be crucial for urination volume analysis. After feature analysis, ML techniques can be applied to extract information and predict urination process metrics such as but not limited to voided volume, voiding time, average flow rate (Q_{avg}), maximum flow rate (Qₘₐₓ), abnormal flow patterns), and/or one or more urological diseases and/or symptoms, among others.

In various embodiments, the following step can be executed using sequence-friendly neural network architecture, for instance, but not limited to recurrent neural network sequences to sequence encoders and transformers with an attention mechanism. Large language models are also capable of being used as a part of predictive pipelines due to the ability to analyze structured or tabular data and summarize the results.

In various embodiments, the ML training process can be executed using an artificial voiding data set obtained using a flow generator as shown in FIG. 8. Part of the data set (e.g. 200 or 2000 voiding episodes) can be used for training and the rest for validation.

In various embodiments, both supervised and unsupervised methods can be used to build a predictive model, including few-shot and zero-shot learnings. No matter which method is used for the urination pattern, obtaining the robustness and accuracy evaluation or prediction optimization can be done by comparing the actual urination pattern curve with the predicted curve by calculating the error, for instance, but not limited to mean squared error, mean average error for each data point or for important signal parts. In addition, to the error metrics mentioned above the curve itself can be used to optimize important parameters like total voided volume, voiding time, average flow rate (Q_{avg}), maximum flow rate (Qₘₐₓ), and abnormal flow patterns. In most cases, the optimization process is iterative.

The result of the optimization is represented as an algorithm kernel and weights. They are stored in the persistent form in a computational environment. This predictive model should be used for final parameters extraction using the raw signal from sensors.

Processing the information from these sensors is often accompanied by the use of special pattern recognition algorithms, in particular those typical of machine learning (“ML”) technologies, which are used to determine the amount and classification of chemicals detected by the above-mentioned sensors. Such algorithms and mathematical models include, without limitation, pattern recognition algorithms, principal component analysis (PCA), linear discriminant analysis (LDA), support vector machines (SVM), artificial neural networks (ANN), and deep learning.

According to embodiments of the invention, adjusting, refining, or tuning of the system can take place according to the following algorithm. When training the system, the user interacts with the system, each time using various technical means, e.g., presses a button and thereby provides the system with information (control signal) affirming or denying that the taken biomaterial sample is identified by the optical sensor and associated with the user. Using machine learning technologies (supervised and/or unsupervised), the system is trained to separate the samples collected from different users, and after a certain number of cycles the training process ends and the operational stage begins. At this stage, the system itself determines the belonging of a sample to a specific user by comparing the chemical characteristics of the new sample with the information about the chemical and/or wave characteristics in the one or more databases 136 of the device for receiving, transmitting and storing data (which may be referral values that have been previously established, for example, by machine learning techniques). The formation of a database 136, which contains data on the chemical or wave characteristics of samples of a biomaterial of a particular subject, is carried out by creating an initial record of the flow characteristics of samples of a subject's biomaterial sample 112.

With continued reference to FIG. 1, system 100 for includes a computing node 124 that may further include an identification module embedded or electrically coupled thereto. The identification module may use the extracted at least first feature as an input. Identification may use non-learning-based (direct) methods by calculating distances measured from pairs of feature vectors directly or use learning-based methods to further transform the feature vectors before calculating a similarity score. A similarity score can be calculated iteratively between one or more features and/or feature vectors.

Computing node 124 may be communicatively coupled to at least one database 136. Database 136 may be one or more electronic storage systems with stored feature retrievably stored therein. Database 136 may be an organized collection of data stored and accessed electronically. According to embodiments, small databases can be stored on a file system, while large databases are hosted on computer clusters or cloud storage, computing node 124 may utilize one or both of these arrangements. The design of database 136 may include formal techniques and practical considerations including data modeling, efficient data representation and storage, query languages, security and privacy of sensitive data, and distributed computing issues including supporting concurrent access and fault tolerance.

Database 136 may include elements of stored features associated with the user based on one or more chemical characteristics of samples previously submitted to the system or transferred electronically from one or more medical data systems. Database 136 may include processed and stored features such as feature vectors associated with the flow characteristics of a previously submitted biomaterial sample 112 or other biologically-identifiable data.

Database 136 may include a database management system (DBMS), which is the software that interacts with end users such as computing node 124 and medical personnel or other electronic systems, applications, and the database itself to capture and analyze the data. The DBMS software additionally encompasses the core facilities provided to administer the database. The sum total of the database, the DBMS and the associated applications can be referred to as database 136.

In various embodiments, system 100 may operate based on comparing the obtained biomaterial samples 112 of the user with referral data that is stored in the system database 136 (the so-called primary sampling of a subject's biomaterial) and which takes into account the characteristics of the uroflowmetry of the biomaterial (urine) and the process of identifying urological diseases and symptoms of a person based on the comparison of the specified data and, as a result, ensuring the complete automation of the medical diagnostics process.

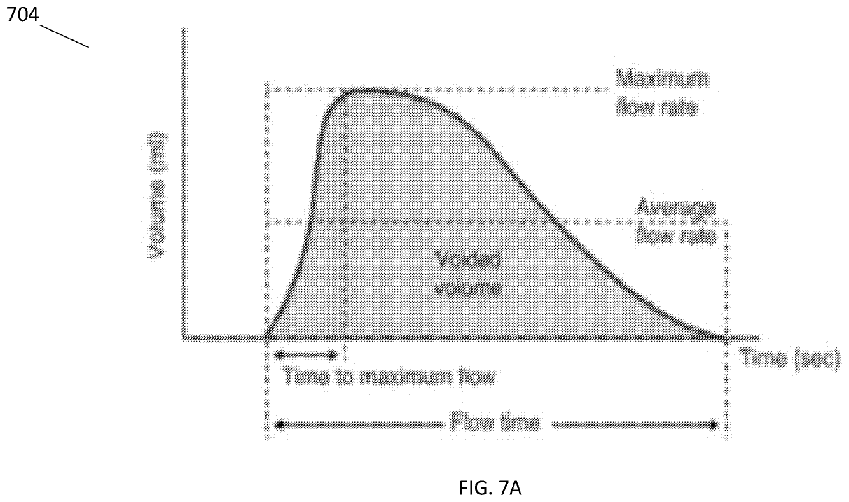

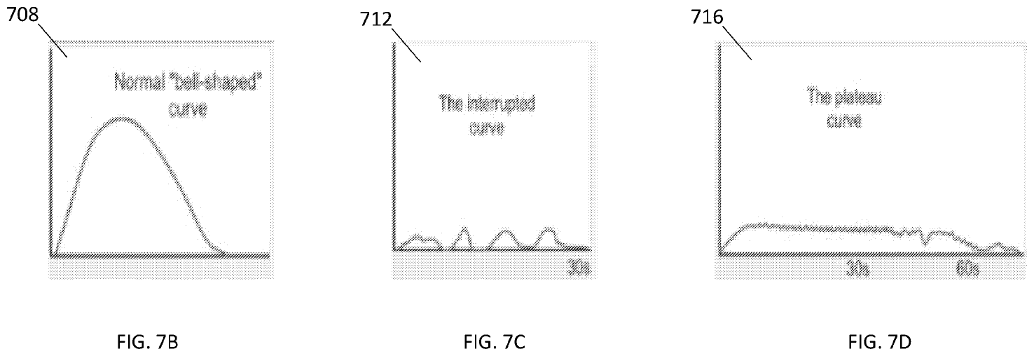

Still referring to FIG. 1, computing node 124 is configured to generate, in response to the parameter 120 of biomaterial sample 112, an electronic diary entry 128 corresponding to the parameter 120. In various embodiments, the computing node 124 is configured to generate an electronic diary entry 128 in response to the signal from the sensor 116, the electronic diary entry 128 having a datum corresponding to the signal. Electronic diary entry 128 may be one or more electronic records representative of the detected or measured parameter 120 associated with the biomaterial sample 112. Electronic diary entry 128 may be stored locally in one or more memories of the computing node 124, the database 136 or offsite in a similar structure. In various embodiments, electronic diary entry 128 may represent the time of day the user has urinated, period of urination, representation of the flow or one or more parameters thereof, or the like. In various embodiments, electronic diary entry 128 may represent the number of urinations per period (e.g., day/night/weeks/etc.), automatically creating a bladder diary or voiding diary. The voiding diary may be automated transmitted to one or more medical providers, a user device, computing device, screen or other API. In various embodiments, voiding diary may be stored in one or more memories or databases 136. Electronic diary entry 128 may be representative of any number of urological diseases or symptoms detected or predicted by the system 100. In various embodiments, electronic diary entry 128 may be stored as data such as plots in FIGS. 7A-7D. In various embodiments, electronic diary entry 128 may be presented to the user via one or more user devices, such as the smartphone on an application. In various embodiments, the electronic diary entry 128 may be presented by PDF to the user. In various embodiments, electronic diary entry 128 may be presented to the doctor via an API integration with a doctor's computer system. In various embodiments, computing device 124 may be configured to generate or record a broad range of information including IPPS symptoms, uroflowmetry dynamics or data points and the bladder diary.

With continued reference to FIG. 1, computing device 124 may be configured to generate a probability score 132 in response to the parameter 120. In various embodiments, probability score 132 may form a part of an International Prostate Symptoms Score (IPSS). In various embodiments, probability score 132 may be formed externally from IPSS. In various embodiments, probability score 132 may be generated in response to detection of flow metrics of a urine stream—such as the probability the user has a urological disease or condition based on symptoms that have been measured or predicted to occur based on the uroflowmetry. In various embodiments, probability score 132 may be transmitted to the user via a screen or API. In various embodiments, probability score 132 may be transmitted visually or audibly to the user. In various embodiments, probability score can identify a risk of the user having a disease or symptom that may lead to an illness or condition, including urological and/or diabetes related diseases. For instance, by measuring total voided volume and urine speed during a week, the system 100 can output (with a probability) that current treatment strategy is not effective or that there is a risk of acute urine retention leading to an emergency room visit. This probability score, depending on the use case, can be reported to a doctor, patient and/or caregiver or any other role in the system. Probability score can be influenced by the information input by, or about the user. For example and without limitation, if a doctor or user reports that the user drank more cups of coffee than usual, then an increased number of urinations may not be indicative of a urological diseases and symptoms. In various embodiments, external information, which may also be obtained from one or more third party systems, including other devices, to adjust the probability score.

In various embodiments, probability score 132 may be transmitted to one or more medical professionals, medical providers, doctors, hospitals or exterior users. In various embodiments, probability score 132 may be transmitted to the user device such as a computer, laptop or smartphone. In various embodiments, the probability score 132 may be transmitted over a wireless cellular network, internet connection, Bluetooth connection. In various embodiments, system 100 may transmit results to the user in an electronic mail (email), text message, SMS, or automated telephone call. Computing node 124 may automatically assess LUTS, including frequency of urination, nocturia, and hesitancy (urgency or delay prior to urination), and provide a probability score for each parameter 120. Data can be aggregated and presented for various needed time periods. For example and without limitation, an electronic diary entry 128 may be collected over 3 or 5 day increments. For example and without limitation, an electronic diary entry 128 may be collected over any period of time, including for the operational life of system 100, or a predetermined amount of time by the user or the medical provider.

In various embodiments, computing node 124 may be configured to transmit one or both of the electronic diary entry 128 and probability score 132 to the user device, such as a smartphone, database 136 or a medical provider. In various embodiments, computing node 124 may be configured to generate a report such a medical report and wellness report, the probability score 132 and electronic diary entry 128 may form a portion. In various embodiments, the reports may be generated after urination or voiding event, after a period of time regardless of voiding events, after a certain threshold of voiding events, or upon detection or measurement of a parameter 120 that led to a probability score 132 or electronic diary entry 128 above a certain threshold or contained information flagged by a medical professional. In various embodiments, the computing node 124 may generate one or more reports, probability scores 132 or electronic diary entries 128 on a regular basis or in response to an on-demand request from a third party computing/IT system or authorized user, such as the doctor or medical professional.

The system 100 may be configured to integrate with various interfaces for results presentation. Medical (wellness) reports and any other data can be transferred/demonstrated to the user or the doctor via a smartphone application, desktop application, web application, chatbot message, email, text message or the like. In various embodiments, the one or more reports may be transmitted in PDF or another text-based file format. The one or more reports may include the probability score associated with a disease or symptom, and can advise the user to drink more water. In various embodiments, the one or more reports may train the user to increase urination time. Also it can advise a person to revisit a doctor, change therapy or change certain related behaviors (beverage intake). The user can additionally input some information about their internal sensations or feelings, such as how they estimate their ability to empty bladder, urgency or provide a quality of life score before, during and after using the system.

Referring now to FIG. 6, an embodiment of a sensor suite 600 which may be utilized in system 100 as described herein. Sensor suite 600 may include an ID sensor 118, the ID sensor 118 being the same or similar to the ID sensor 118 as described herein. For example and without limitation, ID sensor 118 may be a proximity sensor. ID sensor 118 may be disposed on the sensor suite 600 which is proximate the sampling reservoir 104 and configured to have a field of view surrounding the sampling reservoir 104. In various embodiments, ID sensor 118 may be emplaced on the sensor 116, on the sampling reservoir 104, itself, or another portion of system 100. In various embodiments, ID sensor 118 may be configured to detect the user approaching the sampling reservoir 104. In various embodiments, ID sensor 118 may be configured to generate a signal in response to detection of a user. ID sensor 118 may transmit said signal to one or more other components of system 100, namely sensor 116 in response to the detection. In various embodiments, ID sensor 118 may generate a signal in response to the detection, the signal configured to initiate a function of the component, for example and without limitation, activate sensor 116, activate computing node 124, or store the detection as data within database 136. In various embodiments, the ID sensor 118 may generate a signal in response to the detection, the signal configured to initiate another system not described herein or in communication with the system 100. In various embodiments, ID sensor 118 may be configured to receive identification information via one or more user inputs, such as buttons, fingerprints, voice commands, or measured parameters 120 (via sensor 116 or directly measured via ID sensor 118). ID sensor 118 may detect interaction with, or be a remote control with buttons, a mobile app that scans a QR code a doctor has provided the user, smart watches, finger print scanner, and/or a camera, among others.

Sensor suite 600 may include sensor 116 consistent with any sensor as described herein. As can be seen in FIG. 6, sensor 116 may be disposed at a distal end of sensor suite 600, the elongate portion of sensor suite disposed therebetween configured to mount the rim of a toilet bowl. Sensor suite 600 may include integrated user guides which may guide the user via a plurality of possible means e.g., by blinking LEDs, smartphone applications, or sound commands. For example and without limitation, system 100 may visually indicate to user via visual device 604, which may be blinking, colored, or otherwise illuminated LEDs, as shown in FIG. 6. For example and without limitation, visual device 604 may light up a specified color such as green, to indicate the user may begin. In various embodiments, visual device 604 may blink in a pattern or illuminate in general to indicate to the user to begin, stop or perform another action, such as input user information to an API.

Referring now to FIG. 7A-7D, various plots of biomaterial sample deposition into sampling reservoir is shown. Specifically, the plots depict urine flows and various parameters associated therewith. Referring specifically to FIG. 7A, the plot 704 shows flow time on the x-axis and volume on the y-axis. As can be seen qualitatively from the plot, the volume of the flow increases sharply to a maximum and gradually trails off over time. The maximum flow rate, time to maximum flow, average flow rate, total flow time, and voided volume can all be measured or detected and visualized via this plot. Any of these values, among others, may represent a detected and/or measured parameter 120. Plot 704 may represent a normal urine flow from a healthy user, or at least a user lacking urological diseases or symptoms. In various embodiments, plot 704 may be presented to the user, transmitted in one or more reports, electronic diary entries 128, probability scores 132, or the like, to the user, database or memory, or a medical professional.

Referring now to FIGS. 7B-7D, specifically, plot 708 plots a normal “bell-shaped” curve that is similar to the plot 704 in FIG. 7A. Plot 708 may show a normal voiding episode wherein the user urinates normally or substantially normally. Plot 712 shows an interrupted curve, wherein the user voids urine but periodically stops. Plot 716 shows the plateau curve, wherein the user voids urine at a constant and sub-maximal volume or flow rate. Plots 704, 708, 712, 716 may be included in one or more generated reports, and/or electronic diary entries 128. Plots 704, 708, 712, 716 may be presented to the user on a screen, over email, textual message, in an automated message or audio message, and/or be sent to a medical professional.

Referring now to FIG. 8, a schematic representation of a system 800 configured to calibrate and/or train system 100 by simulating a urine stream to form a training data set is depicted. In various embodiments, system 800 may include one or more buttons, switches, or other input devices configured to switch the system between calibration mode, training mode and operating mode. For example and without limitation, any system herein may include the switchable modes through a remote control having buttons assigned to each mode. System 800 is configured to generate one or more artificial voidings by utilizing water from a water tank 804, controlled by a computing device 808 and urine flow imitator 812. In various embodiments, the flow imitator 912 is a pump in fluid communication with one or more hoses configured to emulate a human voiding urine (streaming liquid into the toilet bowl). By imitating a variety of flow regimes and how those flows interact with sampling reservoir 104, a training data set can be generated to improve analysis of voiding episodes in system 100. In various embodiments, the calibration may only requires to input 50 ml in five iterations utilizing a beaker that is provided with the system 100. In various embodiments, the user may be commanded or signaled to adjust one or more angles of the sensors present in the system. For example and without limitation, one or more screens, such as a smartphone may include an angle measurement and command associated with the sensor 116, such as a message indicating the sensor 116 may require to be tilted down 10 degrees.

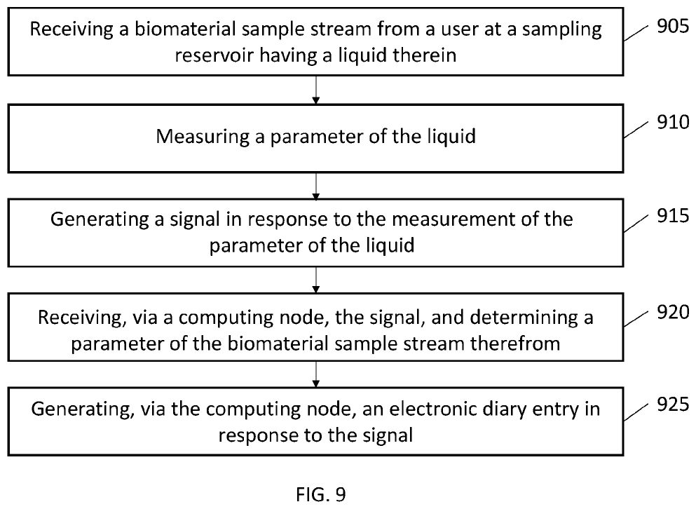

Referring now to FIG. 9, a method 900 for monitoring urological diseases and symptoms is presented in flow chart view. Method 900, at step 905 includes, receiving a biomaterial sample stream from a user at a sampling reservoir having a liquid therein. The biomaterial sample may be any biomaterial sample 112 as described herein. The biomaterial sample 112 may be urine voided from a human user. Sampling reservoir may be any sampling reservoir 104, which may be a toilet bowl. Receiving the biomaterial sample 112 from the user may include the user voiding urine into the liquid 108 held within the sampling reservoir 104. For example and without limitation, the user may stand proximate the toilet, and void urine into said toilet to initiate the measurement process. In various embodiments, the liquid 108 is water. In various embodiments, the liquid 108 is a liquid with a known viscosity and density.

Method 900, at step 910 includes, measuring a parameter of the liquid within the sampling reservoir via at least one sensor. The sensor may be any sensor 116 and/or ID sensor 118 as described herein. In various embodiments, the sensor is submerged in the liquid within the sampling reservoir. In various embodiments, the sensor is a time-of-flight (TOF) sensor. In various embodiments, the sensor may be a triangularity sensor. In various embodiments, the one or more sensors may be coupled to the toilet bowl, toilet tank, or disposed exterior to the toilet and proximate thereto. In various embodiments, the parameter of the liquid may be an intensity of a wave formed therein. In various embodiments, the parameter of the liquid may be amplitude or height of the waves created in the liquid from the urine stream. In various embodiments, the parameter of the liquid is an amplitude of at least one wave formed in the liquid. In various embodiments, the at least one wave is formed in the liquid in response to deposition of the biomaterial sample into the liquid, such as for example, urination into the toilet bowl. In various embodiments, the amplitude of the at least one wave measured by the sensor is correlated with an urination flow pattern.

In various embodiments one or more sensors, namely sensor 116 and/or ID sensor 118 may detect the user approaching the sampling reservoir. In various embodiments, the sensor 116 or the ID sensor 118 may be a proximity sensor. In various embodiments, the sensor 116 or the ID sensor 118 may include a proximity sensor amongst other types of sensors in a sensor suite, as described in conjunction with sensor suite 600. In various embodiments, the proximity sensor is configured to transmit an activation signal to the sensor in response to the detection of the user proximate the sampling reservoir. In various embodiments, system 100 and/or sensor 116 and ID sensor 118 may include an identification component configured to identify the user. In various embodiments the identification component may be a fingerprint sensor.

With continued reference to FIG. 9, method 900 includes, at step 915, generating a signal in response to the measurement of the parameter the liquid within the sampling reservoir. In various embodiments, one or more sensors, such as sensor 116 and ID sensor 118 may generate an electrical signal representative of a detection event or measurement of a parameter of the urine stream and liquid within the sampling reservoir. In various embodiments the signal from the sensor is transmitted to a computing node via wireless connection.

With continued reference to FIG. 9, method 900 includes, at step 920, receiving, via the computing node, the signal from the sensor and determining therefrom, a parameter of the biomaterial sample. In various embodiments the parameter of the biomaterial sample is a flow rate. In various embodiments, the parameter of the biomaterial sample is a voided volume. In various embodiments, the parameter of the biomaterial sample is a void time. In various embodiments, the parameter of the biomaterial sample is a void frequency. In various embodiments, the computing node is any computing node 124 as described herein or otherwise. In various embodiments, the sensors and computing node are connected via a wireless connection over a cellular network, WiFi or Bluetooth connection, or over the internet. In various embodiments, the sensors and computing node are connected via a wired connection, for example utilizing a USB connection. In various embodiments, computing node and sensors may be communicatively connected to a common component, the common component configured to facilitate communication therebetween or between these components and any exterior computing devices. In various embodiments, the computing node 124 is configured to extract data from the signal, the data extraction may include a feature extraction as described herein. Receiving the signal from the sensors may include performing one or more data extractions and or features extraction consistent with the entirety of this disclosure. In various embodiments, computing node may include one or more ML algorithms configured to perform feature extraction and/or predict one or more diseases or symptoms of a user.

With continued reference to FIG. 9, method 900 includes, at step 925, generating, via the computing node, an electronic diary entry in response to the signal. In various embodiments, the electronic diary entry is transmitted to a user device. In various embodiments, the user device is a smartphone. In various embodiments, the feature extraction further includes machine learning (ML) based information extraction. In various embodiments, the computing node may be configured to generate a probability score in response to the signal. In various embodiments, the probability score corresponds to a probability the user has a urological disease.

In various embodiments, the computing node 124 is configured to generate an electronic diary entry 128 in response to the signal from the sensor 116, the electronic diary entry 128 having a datum corresponding to the signal. Electronic diary entry 128 may be one or more electronic records representative of the detected or measured parameter 120 associated with the biomaterial sample 112. Electronic diary entry 128 may be stored locally in one or more memories of the computing node 124, the database 136 or offsite in a similar structure. In various embodiments, electronic diary entry 128 may represent the time of day the user has urinated, period of urination, representation of the flow or one or more parameters thereof, or the like. In various embodiments, electronic diary entry 128 may represent the number of urinations per period (e.g., day/night/weeks/etc.), automatically creating a bladder diary or voiding diary. The voiding diary may be automated transmitted to one or more medical providers, a user device, computing device, screen or other API. In various embodiments, voiding diary may be stored in one or more memories or databases 136. Electronic diary entry 128 may be representative of any number of urological diseases or symptoms detected or predicted by the system 100. In various embodiments, electronic diary entry 128 may be stored as data such as plots in FIGS. 7A-7D.

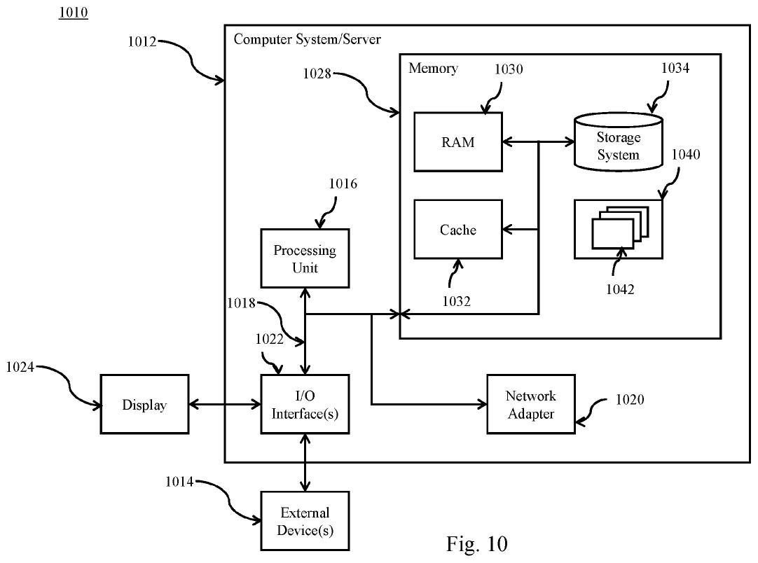

Referring now to FIG. 10, a schematic of an example of a computing node is shown. Computing node 1010 is only one example of a suitable computing node and is not intended to suggest any limitation as to the scope of use or functionality of embodiments described herein. Regardless, computing node 1010 is capable of being implemented and/or performing any of the functionality set forth hereinabove.

In computing node 1010 there is a computer system/server 1012, which is operational with numerous other general purpose or special purpose computing system environments or configurations. Examples of well-known computing systems, environments, and/or configurations that may be suitable for use with computer system/server 1012 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 1012 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 1012 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 10, computer system/server 1012 in computing node 1010 is shown in the form of a general-purpose computing device. The components of computer system/server 1012 may include, but are not limited to, one or more processors or processing units 1016, a system memory 1028, and a bus 1018 that couples various system components including system memory 1028 to processor 1016.

Bus 1018 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 1012 typically includes a variety of computer system readable media. Such media may be any available media that is accessible by computer system/server 1012, and it includes both volatile and non-volatile media, removable and non-removable media.

System memory 1028 can include computer system readable media in the form of volatile memory, such as random access memory (RAM) 1030 and/or cache memory 1032. Computer system/server 1012 may further include other removable/non-removable, volatile/non-volatile computer system storage media. By way of example only, storage system 1034 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 1018 by one or more data media interfaces. As will be further depicted and described below, memory 1028 may include at least one program product having a set (e.g., at least one) of program modules that are configured to carry out the functions of embodiments of the disclosure.

Program/utility 1040, having a set (at least one) of program modules 1042, may be stored in memory 1028 by way of example, and not limitation, as well as an operating system, one or more application programs, other program modules, and program data. Each of the operating system, one or more application programs, other program modules, and program data or some combination thereof, may include an implementation of a networking environment. Program modules 1042 generally carry out the functions and/or methodologies of embodiments as described herein.

Computer system/server 1012 may also communicate with one or more external devices 1014 such as a keyboard, a pointing device, a display 1024, etc.; one or more devices that enable a user to interact with computer system/server 1012; and/or any devices (e.g., network card, modem, etc.) that enable computer system/server 1012 to communicate with one or more other computing devices. Such communication can occur via Input/Output (I/O) interfaces 1022. Still yet, computer system/server 1012 can communicate with one or more networks such as a local area network (LAN), a general wide area network (WAN), and/or a public network (e.g., the Internet) via network adapter 1020. As depicted, network adapter 1020 communicates with the other components of computer system/server 1012 via bus 1018. It should be understood that although not shown, other hardware and/or software components could be used in conjunction with computer system/server 1012. Examples, include, but are not limited to microcode, device drivers, redundant processing units, external disk drive arrays, RAID systems, tape drives and data archival storage systems, among others.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1-59. (canceled)

60. A system for monitoring urological symptoms, the system comprising:
a sampling reservoir comprising an opening and having a liquid held therein, the sampling reservoir configured to receive a biomaterial sample from a user through the opening;
a first sensor disposed proximate to the sampling reservoir, the first sensor configured to:
measure a parameter of the liquid; and
generate a signal in response to the measurement; and
a computing node, the computing node configured to receive the signal from the first sensor, and determine therefrom a parameter of the biomaterial sample.

61. The system of claim 60, wherein the parameter of the liquid is an amplitude of at least one wave formed in the liquid.

62. The system of claim 61, wherein the at least one wave is formed in the liquid in response to deposition of the biomaterial sample into the liquid, and wherein the amplitude of the at least one wave is correlated with a urination flow pattern.

63. The system of claim 60, wherein the sampling reservoir is a toilet bowl.

64. The system of claim 60, wherein the biomaterial sample is urine voided from the user.

65. The system of claim 60, wherein the computing node is further configured to generate an electronic diary entry in response to the signal from the first sensor, the electronic diary entry having a datum corresponding to the signal.

66. The system of claim 60, wherein:
the computing node is configured to extract one or more features from the signal, wherein the one or more features comprises a void frequency and provide the one or more features to a machine learning (ML) model.

67. The system of claim 66, wherein the parameter includes one or more of a flow rate, a voided volume, a flow pattern, and a urination time.

68. The system of claim 60, wherein the computing node is configured to generate a probability score in response to the signal from the sensor, wherein the probability score corresponds to a probability the user has a disease.

69. The system of claim 60, further comprising an identification component, wherein the identification component is configured to identify the user.

70. The system of claim 69, wherein the identification component is further configured to re-identify the user based on urination patterns.

71. A method for monitoring urological diseases and symptoms, the method comprising:
receiving a biomaterial sample stream from a user at a sampling reservoir having a liquid therein;
measuring a parameter of the liquid within the sampling reservoir via at least one sensor;
generating a signal in response to the measurement of the parameter of the liquid within the sampling reservoir;
receiving, via a computing node, the signal from the at least one sensor and determining therefrom a parameter of the biomaterial sample stream; and
generating, via the computing node, an electronic diary entry in response to the signal.

72. The method of claim 71, wherein the parameter of the liquid is an amplitude, correlated with a urination flow pattern, of at least one wave that is formed in the liquid in response to deposition of the biomaterial sample into the liquid.

73. The method of claim 71, further comprising:
extracting, via the computing node, one or more features from the signal, the one or more features comprising a void frequency;
providing, via the computing node, the one or more features to a machine learning (ML) model.

74. The method of claim 73, wherein parameter includes one or more of a flow rate, a voided volume, a flow pattern, and a urination time.

75. The method of claim 71, wherein the sampling reservoir is a toilet bowl.

76. The method of claim 71, wherein the biomaterial sample is urine voided from the user.

77. The method of claim 71, further comprising identifying the user via an identification component.

78. The method of claim 71, wherein the electronic diary entry is transmitted to a user device.

79. The method of claim 78, wherein the identification component is further configured to re-identify the user based on urination patterns.
